(19) United States
(12) Patent Application Publication
(10) Pub. No.: US 2024/0238249 A1
(43) Pub. Date: Jul. 18, 2024

(54) Lipidic Nano-Pharmaceuticals

(71) Applicant: Huxley Health Inc., Concord (CA)

(72) Inventors: John Trant, Windsor (CA); Jonathan Simone, Kleinburg (CA); Abhinandan Banerjee, Windsor (CA); John Hayward, Windsor (CA); Anton Homon, Windsor (CA)

(21) Appl. No.: 18/395,040

(22) Filed: Dec. 22, 2023

Related U.S. Application Data
(60) Provisional application No. 63/434,697, filed on Dec. 22, 2022.

(51) Int. Cl. A61K 31/4045; A61K 31/675; A61K 47/18

(52) U.S. Cl. CPC A61K 31/4045; A61K 31/675; A61K 47/18

(57) ABSTRACT

Disclosed are various embodiments for increasing the biotransport characteristics of a psychedelic active ingredient across the mammalian blood-brain barrier. Disclosed are pharmaceutically-acceptable psychedelics that, when administered, convert to an active form in vivo, and act as a 5HT_{2A}R agonist which are useful to treat psychiatric disorders, neurological disorders, degenerative disorders, inflammatory disorders, and symptoms thereof.

### FIELD OF THE INVENTION

The invention belongs to the field of new auxiliary materials and new dosage forms of medicinal preparations and comprises synthesis of psychedelic prodrugs conjugated to lipidic moieties. Such moieties may include fatty acids (such as oleic, linoleic, undecanoic, or stearic acid); glycerides; or phospholipids. The psychedelic may be conjugated to the lipidic appendage with or without spacers such as amino acids, PEG or self-immolative polymers.

### BACKGROUND OF THE INVENTION

Tryptamine is an indolamine metabolite of the essential amino acid tryptophan. The structure of tryptamine is a share feature of certain aminergic neuromodulators including melatonin, serotonin, bufotenin as well as psychedelic derivatives such as dimethyltryptamine (DMT), psilocybin, and psilocin. There remains a need in the art for tryptamine analogue-based medicines.

Psilocin, the active drug derived from prodrug psilocybin, and other 5-hydroxytryptamine receptor 2A (5HT_{2A}R) agonists are classic psychedelics traditionally used in religious, spiritual, and ritualistic contexts within indigenous cultures. Despite some initial interest in tryptamines as probes of brain function and as experimental therapeutics, the prohibition culture of the 20th century led to their use and possession being criminalized, effectively halting research. However, there has been a re-emergence of interest as preliminary reports suggesting they may prove as or more beneficial than the pharmacological standard-of-care for conditions related both directly—depression, anxiety, depersonalization, and obsessive-compulsive disorder—and indirectly—obesity, migraine, alcohol- and drug-dependence—to mood and behavioral disorders. The nascent nature of the field both complicates the research, as there is little to build from, but also, of course, provides very significant opportunity for innovation.

Over 100 species of mushrooms have been found to contain psilocybin. Low physiological toxicity and low abuse liability (so demonstrated by marginal levels of drug self-administration in rhesus monkeys of psilocybin, and psilocin, make them promising therapeutic candidates. Mescaline (3,4,5-trimethoxyphenethylamine) is another naturally occurring psychedelic proto-alkaloid of the substituted phenethylamine class, derived primarily from cacti native to the Americas. Mescaline has a wide array of suggested medical uses, including treatment of alcoholism and depression, due to these disorders having links to serotonin deficiencies.

Microdosing, use of a low dose below the perceptual threshold, has been claimed to not impair ‘normal’ functioning of an individual while improving their well-being and enhancing their cognitive and/or emotional processes. The exact doses involved in such practices can often be poorly defined, and microdosing schedules have been determined on the basis of anecdotal evidence, with very few scientific studies on the intricacies associated with the microdosing regimen. Consuming natural sources directly, such as dried P. cubensis ('Shrooms), although simple, does not provide every individual with the same dose, and can vary from batch to batch. Lindenblatt et al. revealed a large interindividual variation as regards psilocin plasma concentrations in healthy volunteers after oral administration of identical dosages of psilocybin. This likely arises from differential uptake from the intestine, clearance, and processing in the liver. This is not ideal when the goal is microdosing to accompany psychotherapy. This patent application reveals a series of lipidic psychedelic prodrugs of psylocibin, psilocin, ibogaine, mescaline, ibogaine and iboga-derived alkaloids, mescaline, LSD, MDMA, members of the 2-C class of phenethylamines, psychedelic tryptamines, phenethylamines, indole alkaloids and related compounds with three unique properties:
(i) reduced-loss delivery platforms for accurate and precise dosing of psychedelics;
(ii) enhanced lymphatic uptake of psychedelics to avoid first pass metabolism effects; and
(iii) targeted transporting of psychedelics across the blood brain barrier (BBB).

Self-immolative polymers are an emerging class of macromolecules with distinct disassembly profiles that set them apart from other general degradable materials. These polymers are programmed to disassemble spontaneously from head to tail, through a domino-like fragmentation, upon response to extremal stimuli. In the time since we first reported this unique type of molecule, several groups around the world have developed new, creative molecular structures that perform analogously to our pioneering polymers. Self-immolative polymers are now widely recognized as an important class of stimuli-responsive materials for a wide range of applications such as signal amplification, biosensing, drug delivery, and materials science. According to the present disclosure, such materials can be an effective tool to achieve rapid domino-like fragmentation of polymeric molecules that are used for the delivery of psychedelic active ingredients. See Shelef et al., “Self-Immolative Polymers: An Emerging Class of Degradable Materials with Distinct Disassembly Profiles”, J. Am. Chem. Soc., December 2021, 143, 50, 21177-21188.

It would be desirable to have greater options for pharmaceutically effective and acceptable tryptamine analogue-based medicines.

In particular, it would be desirable to provide therapeutically effective aminergic neuromodulators including melatonin, serotonin, bufotenin as well as psychedelic derivatives such as dimethyltryptamine (DMT), psilocybin, and psilocin.

It would also be desirable to have a method for administering to a subject in need thereof a therapeutically effective amount of an acceptable tryptamine analogue in a pharmaceutically acceptable carriers or excipients, wherein the subject is a mammal and/or a human.

### SUMMARY OF THE INVENTION

The present invention describes prodrugs and the selection of pro-drug strategies that are uniquely suitable for psychedelic compounds, especially tryptamine analogues such as those derivatives having psychedelic effects (e.g., dimethyltryptamine, psilocybin, and psilocin) as APIs.

The present invention concerns pharmaceutically-acceptable psychedelics, tryptamine analogues and salts thereof. In particular, though not exclusively, the invention relates to formulations and uses of the same as a medicament. When administered, these materials convert to an active form in vivo, and act as a 5HT_{2A}R agonist which are useful to treat mental disorders, such as a depressive condition, including unipolar and bipolar depressive conditions, such as, e.g., depression, depression from generalized anxiety, major depression, treatment resistant depression and postpartum depression.

In another aspect, the invention comprises a method of treating a mental disorder, comprising the step of administering an effective amount of a compound described herein. In some embodiments, the mental disorder is a depressive condition, including unipolar and bipolar depressive conditions, such as but not limited to depression, depression from generalized anxiety, major depression, treatment resistant depression and postpartum depression.

The invention provides for the treatment and/or prevention of psychiatric disorders, and/or neurological disorders, and/or degenerative disorders, and/or inflammatory disorders.

In another aspect, the invention relates to the use of a compound described herein to treat a mental disorder, or in the manufacture of a medicament for treating a mental disorder, such as depression.

The present invention is directed to compositions and methods for the treatment of a variety of mental disorders, including depression and depressive symptoms, with a method that comprises: (a) administering an effective amount of a composition comprising a psychedelic prodrug having a structure according those shown in the following table:

| | |
|---|---|
| 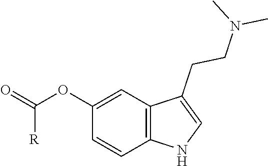 | A |
| 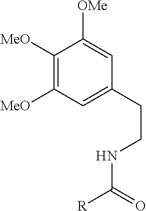 | A′ |
| 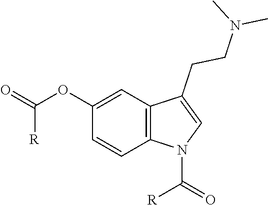 | A″ |
| 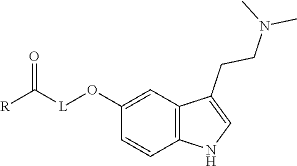 | B |
| 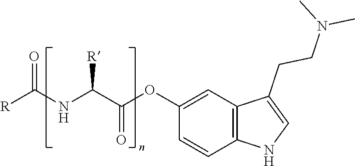 | B′ |
| 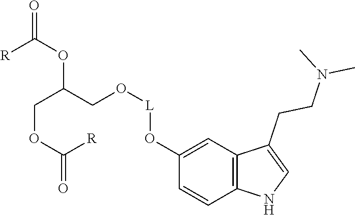 | C |
| 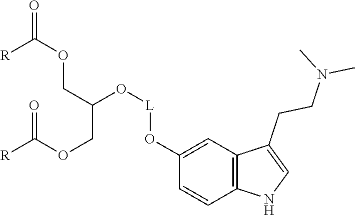 | C′ |
| 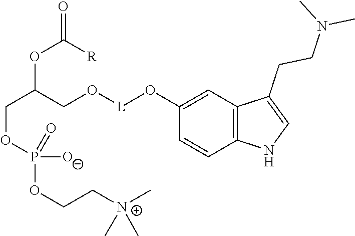 | D |
| 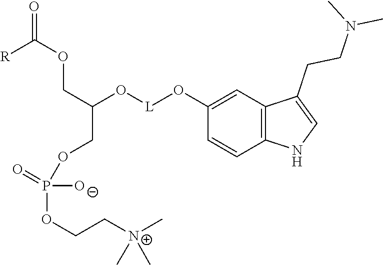 | D′ |
| 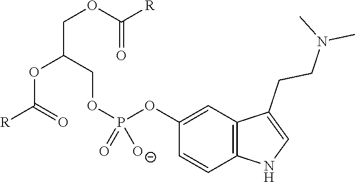 | E |
| 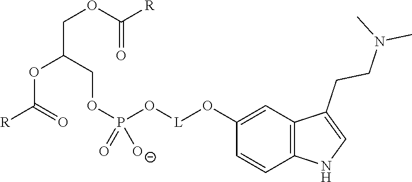 | E′ |
| 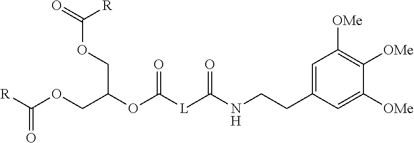 | F |
| 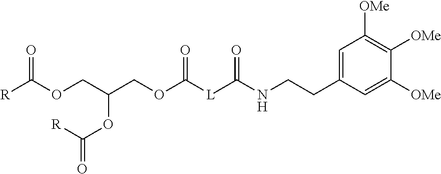 | F′ |
| 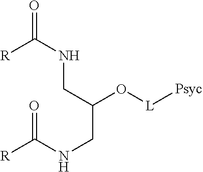 | G |
| 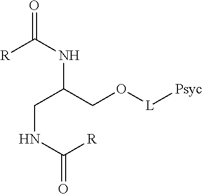 | |
| 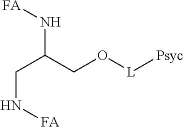 | G′ |
| 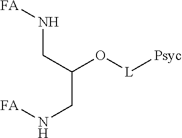 | |

wherein:
A represents active psychedelic ingredients functionalized through an amide, ester, carbonate, carbamate, thiocarbamate or thiocarbonate group to fatty acids and other groups imparting lipophilicity, wherein the psychedelics are functionalized on an oxygen atom; on a nitrogen atom; or on both oxygen and nitrogen atoms, and optionally wherein the lipophilicity-imparting group is removable when exposed to a particular stimulus;
B represents active psychedelic ingredients functionalized as in A, A′, or A″, connected through a linker, L, that is (a) monomeric, (b) oligomeric, or (c) polymeric; and is attached to the psychedelic to the lipophilic group by any of ester, carbonate, carbamate, thiocarbamate or thiocarbonate groups;
C represents active psychedelic ingredients linked through any of amide, ester, carbonate, carbamate, thiocarbamate or thiocarbonate to a self-immolative polymer or other 4 to 40 carbon atom spacer connected to a glyceride through a primary or secondary hydroxyl group;
D represents active psychedelic ingredients linked through an ester, carbonate, carbamate, thiocarbamate, or thiocarbonate that is linked to a self-immolative polymer, PEG, PVP or other 4 to 40 carbon atom spacer connected to a phospholipid through a primary or secondary hydroxyl group; and
E represents active psychedelic ingredients linked directly or through an amide, ester, carbonate, carbamate, thiocarbamate, or thiocarbonate that is linked to a self-immolative polymer, PEG, PVP or other 4 to 40 carbon atom linker to a phospholipid phosphate group;
F and F′ represent ingestible mescaline glyceride prodrugs;
G and G′ represent amide bio-isosteres of diglycerides as lipidic appendages to a psychedelic active ingredient;
L represents a linker that can be selected from branched or unbranched alkyl, alkenyl, alkynyl, cycloalkyl, heterocycloalkyl, aryl, aralkyl, heteroaryl, heteroaralkyl, consisting of between 4 and 48 atoms;
wherein R groups can be independently and differently selected from branched or unbranched alkyl, alkenyl, alkynyl, cycloalkyl, heterocycloalkyl, aryl, aralkyl, heteroaryl, heteroaralkyl, having between 4 and 48 atoms, wherein the R groups can be differentially substituted; and
wherein the functionality between linker and non-psychedelic terminus in each case includes an amide ester, carbonate, carbamate, thiocarbamate or thiocarbonate, or ether.

The compositions of the present invention show promise as psychedelic prodrugs for the treatment of psychiatric disorders, neurological disorders, degenerative disorders, inflammatory disorders, and symptoms thereof.

### DETAILED DESCRIPTION OF THE INVENTION

The general method of treatment comprises administering to a human or mammal subject in need thereof a therapeutically-effective amount of an acceptable psychedelic analogue in one or more pharmaceutically acceptable carriers or excipients.

The administered composition is formulated for oral, sublingual, intranasal, pulmonary administration, buccal, sublingual, rectal, transdermal, transmucosal, epidural, intrathecal, intraocular topical, creams, lotions, gels and eye drops using one or more excipients that are traditionally used in such formulations.

Embodiments of the present invention comprise novel synthetic psychedelic prodrugs. The prodrugs may be useful for treatment of mental disorders such as depression, including without limitation, major depression, treatment resistant depression and postpartum depression. As used herein, the term “mental disorder” includes those disorders which may be diagnosed by a mental health professional as a psychological or psychiatric disorder, including those which may be diagnosed by reference to Diagnostic and Statistical Manual of Mental Disorders (DSM-5).

The term “treating”, “treat” or “treatment” as used herein embraces both preventative, i.e., prophylactic, and palliative treatment, i.e., relieve, alleviate, or slow the progression of the patient's disease, disorder or condition.

As used herein, “psychedelic state” is an altered state of consciousness experienced by a person, which may include intensified sensory perception, perceptual distortion or hallucinations, and/or feelings of euphoria or despair. Psychedelic states have been described as resulting from psychedelic drugs such as DMT (dimethyltryptamine), LSD, mescaline or psilocybin. Other known psychedelic drugs include the 4-hydroxy analogs of N-Methyl-N-isopropyltryptamine (MiPT) and N,N-diisopropyltryptamine (DiPT).

The present invention comprises prodrugs of 5HT_{2A}R agonists which induce a psychedelic state or which still provide a beneficial therapeutic effect without being associated with a psychedelic state. The prodrugs may be used in combination with other treatments known to be effective for treating mental disorders, such as psychotherapy, electroconvulsive therapy and/or other pharmaceutical compounds, for example, with concomitant use of tricyclic antidepressants (TCAs), selective serotonin reuptake inhibitors (SSRIs), selective norepinephrine reuptake inhibitors (SNRIs), monoamine oxidase inhibitors (MOAIs) or other anti-depressants. In preferred embodiments, the treatment may produce lasting effects, for example longer than 1 month after a single treatment, preferably longer than 3 months, and more preferably longer than 6 months. In some embodiments, additional therapy may not be required.

“Compounds” when used herein includes any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs. The expression “prodrug” refers to compounds that are drug precursors which following administration, release the drug (or “active”) in vivo via some chemical or physiological process (e.g., hydrolysis, enzymatic cleavage or hydrolysis, or metabolism is converted to the desired drug form). The invention includes within its scope the pharmaceutically acceptable salts of the compounds of the invention. Accordingly, the phrase “or a pharmaceutically acceptable salt thereof” is implicit in the description of all compounds described herein unless explicitly indicated to the contrary.

In some embodiments, the compounds of the present invention comprise prodrug compounds that are readily purified, formulated and stable, and preferably may be used to provide highly soluble drug substances, with fast onset and elimination for convenient use in a clinical setting. In some embodiments, the compounds may be produced as a zwitterion, which may be converted to a pharmaceutically acceptable salt.

In some embodiments, the compounds of the present invention preferably allow for fast cleavage in vivo of the prodrug moiety to give the active pharmacophore, for example, 90% conversion may occur in under 4 hours, preferably in less than 2 hours, and more preferably in less than 1 hour. Prodrugs may have lesser, little or no pharmacological activity themselves, however when administered to a patient, may be converted into an active compound, for example, by hydrolytic cleavage.

“Alkyl,” by itself or as part of another substituent, refers to a saturated branched, straight-chain or cyclic monovalent hydrocarbon radical derived by the removal of one hydrogen atom from a single carbon atom of a parent alkane. The term “alkyl” includes cycloalkyl. Typical alkyl groups include, but are not limited to, methyl; ethyl; propyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like. In some embodiments, an alkyl group comprises from 1 to 20 carbon atoms (C1-C20 alkyl). In other embodiments, an alkyl group comprises from 1 to 10 carbon atoms (C1-C10 alkyl). In still other embodiments, an alkyl group comprises from 1 to 6 carbon atoms (C1-C6 alkyl) or 1 to 4 carbon atoms (C1-C4). C1-C6 alkyl is also known as “lower alkyl”.

The term “arylalkyl” is a term of the art and as used herein refers to an alkyl group, for example a C1-6 alkyl group, substituted with an aryl group, where the residue is linked to the main molecule through the alkyl group. An example of arylalkyl is the benzyl group, that is, the phenyl-methyl group.

“Substituted,” when used to modify a specified group or radical, means that one or more hydrogen atoms of the specified group or radical are each, independently of one another, replaced with the same or different substituent(s). The term “substituted” specifically envisions and allows for one or more substitutions that are common in the art. However, it is generally understood by those skilled in the art that the substituents should be selected so as to not adversely affect the useful characteristics of the compound or adversely interfere with its function.

The term “optionally substituted” denotes the presence or absence of the substituent group(s). That is, it means “substituted or unsubstituted”. For example, optionally substituted alkyl includes both unsubstituted alkyl and substituted alkyl. The substituents used to substitute a specified group can be further substituted, typically with one or more of the same or different groups selected from the various groups specified above.

The term “psychiatric disorders” refers to one or more disorders selected from the following psychiatric disease as defined as defined by DMS5 and ICD11 that may benefit from modulation of neural plasticity, including Schizophrenia spectrum and other psychotic disorders, Bipolar and related disorders, Depressive disorders, COVID Depressive disorder, generalized anxiety disorders, Obsessive-compulsive and related disorders, Trauma- and stressor-related disorders, Dissociative disorders, Somatic symptom and related disorders, Feeding and eating disorders, Elimination disorders, Sleep-wake disorders, Sexual Disruptive, impulse-control, and conduct disorders, Substance-related and addictive disorders, panic disorder, agoraphobia, social anxiety disorder, phobias, posttraumatic stress disorder, obsessive compulsive disorder, generalized anxiety disorder, anorexia nervosa, binge eating disorder, bulimia nervosa, psychosis, schizophrenia, substance addiction and personality disorders, neurocognitive disorders, personality disorders, paraphilic disorders and for the reduction of suicidal ideation in a patient suffering from a life-threatening disease.

The term “neurological disorders” refers to any structural, biochemical and/or electrical abnormalities in the brain, spinal cord or other nerves and includes neurodevelopment and neurodegenerative diseases that may benefit from of neural plasticity modulation. In a preferred embodiment, the term “neurological disorder” refers to one or more disorders selected from the following acquired brain injury, ataxia brain tumor, dementia, dystonia epilepsy, temporal lobe epilepsy, pain associated with neurological disorders, headache disorders, functional and dissociative neurological symptoms, neuroinfections, meningitis, disorders associated with malnutrition, motor neuron disease, multi-system atrophy, multiple sclerosis, amyotrophic lateral sclerosis, mesial temporal lobe hippocampal sclerosis, muscular dystrophy, myalgic encephalomyelitis, Parkinson's disease, progressive supranuclear palsy, cerebral palsy, Huntington's disease, Alzheimer's disease, frontal lobe dementia, vascular dementia, dementia with Lewy bodies, mild cognitive impairment (MCI) associated with aging and chronic disease and its treatment, including chemotherapy, immunotherapy and radiotherapy, mild corticobasal degeneration, disorders associated with accumulation of beta amyloid, and/or with the accumulation or disruption of tau protein and its metabolites. Lyme encephalopathy, toxic encephalopathy, cognitive decline associated with aging, spinabifida, hydrocephalus, spinal injury, stroke, Tourette syndrome, and transverse myelitis, corticobasal degeneration, supranuclear palsy, epilepsy; Nervous System trauma, Nervous System infections, Nervous System inflammation, including inflammation from autoimmune disorders, including NMDAR encephalitis, and cytopathology from toxins, (including microbial toxins, heavy metals, and pesticides etc.), stroke, multiple sclerosis, Huntington's disease, mitochondrial disorders, Fragile X syndrome, Angelman syndrome, hereditary ataxias, neuro-otological and eye movement disorders, amyotrophic lateral sclerosis, tardive dyskinesias (TD), hyperkinetic disorders; attention deficit hyperactivity disorder and attention deficit disorders; restless leg syndrome, autism spectrum disorders, tuberous sclerosis, Rett syndrome, cerebral palsy, disorders of the reward system including eating disorders [including anorexia nervosa (“AN”) and bulimia nervosa (“BN”), and binge eating disorder (“BED”), trichotillomania, dermotillomania, nail biting, migraine, fibromyalgia, and peripheral neuropathy of any etiology. Symptoms or manifestations of nervous system disorders that may be treated or prevented by neuroplastogen substances and drugs include, a decline, impairment, or abnormality in cognitive abilities including executive function, attention, cognitive speed, memory, language functions (speech, comprehension, reading and writing), orientation in space and time, praxis, ability to perform actions, ability to recognize faces or objects, concentration, and alertness; abnormal movements including akathisia, bradykinesia, tics, myoclonus, dyskinesias, including dyskinesias relate to Huntington's disease, levodopa induced dyskinesias and neuroleptic induced dyskinesias, dystonias, tremors, including essential tremor, and restless leg syndrome; parasomnias, insomnia, disturbed sleep pattern; psychosis; delirium; agitation; headache; motor weakness, spasticity, impaired physical endurance; sensory impairment, including impairment of vision and visual field defects, smell, taste, hearing and balance, and dysesthesias; dysautonomia; and ataxia, impairment of balance or coordination, tinnitus, neuro-otological and eye movement impairments, neurological symptoms of alcohol withdrawal, including delirium, headache, tremors, hallucinations, hypertension.

The term “degenerative disorders” refers to one or more disorders selected from the following degenerative disorders, neurodegenerative diseases of the retina like glaucoma, diabetic retinopathy and age-related macular degeneration, retinitis pigmentosa, Usher disease and Bardet-Biedl syndrome, motor neuron disease, prion disease, spinocerebelluar ataxia and apathy syndrome.

The term “inflammatory disorders” refers to one or more disorders selected from the following inflammatory disorders, of atherosclerosis, asthma, rheumatoid arthritis, psoriasis, type II diabetes, irritable bowel syndrome, Crohn's disease, septicemia, depression, schizophrenia, multiple sclerosis, conjunctivitis, Alzheimer's disease, chronic obstructive pulmonary disease, neuro-inflammation, metabolic syndrome, impaired glucose tolerance, non-alcoholic fatty liver disease (NAFLD), (NAFL) and their complications, nonalcoholic steatohepatitis (NASH) and conjunctivitis.

The present invention describes prodrugs and the selection of prodrug strategies that are uniquely suitable for psychedelics as APIs. The following tables illustrate the pro-drug embodiments according to the present invention.

A psychedelic prodrug having the structures according to the invention are shown in the table below.

TABLE 1

| | |
|---|---|
| 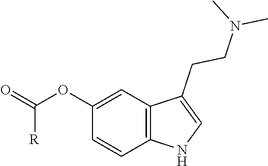 | A |
| 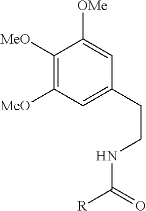 | A′ |
| 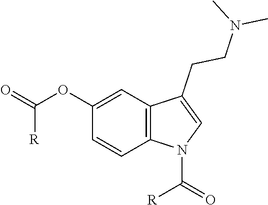 | A″ |
| 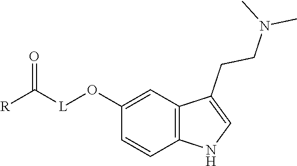 | B |
| 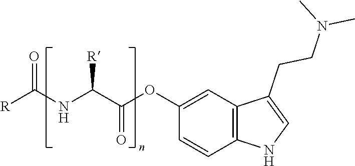 | B′ |
| 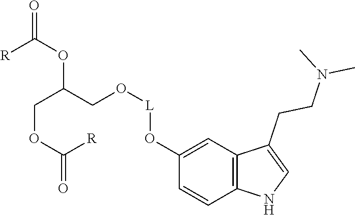 | C |
| 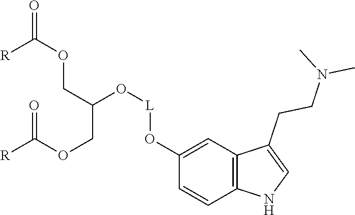 | C′ |
| 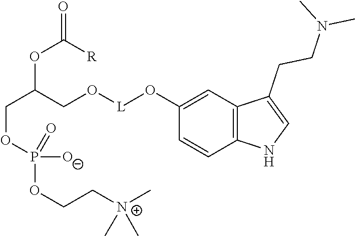 | D |
| 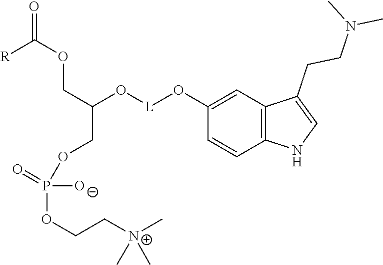 | D′ |
| 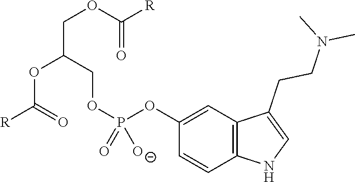 | E |
| 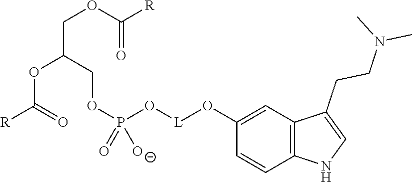 | E′ |

wherein:
A represents active psychedelic ingredients functionalized through an amide, ester, carbonate, carbamate, thioamide, thiocarbamate or thiocarbonate group to fatty acids and other groups imparting lipophilicity, wherein the psychedelics are functionalized on an oxygen atom; on a nitrogen atom; or on both oxygen and nitrogen atoms, and optionally wherein the lipophilicity-imparting group is removable when exposed to a particular stimulus;
B represents active psychedelic ingredients functionalized as in A, A′, or A″, connected through a linker, L, that is (a) monomeric, (b) oligomeric, or (c) polymeric; and is attached to the psychedelic to the lipophilic group by any of amide, ester, carbonate, carbamate, thioamide, thiocarbamate or thiocarbonate groups;
C represents active psychedelic ingredients linked through any of amide, ester, carbonate, carbamate, thioamide, thiocarbamate or thiocarbonate to a self-immolative polymer or other 4 to 40 carbon atom spacer connected to a glyceride through a primary or secondary hydroxyl group, or lipids based on non-glycerin cores such as sphingolipids or ceramides;
D represents active psychedelic ingredients linked through an amide, ester, carbonate, carbamate, thioamide thiocarbamate, or thiocarbonate that is linked to a self-immolative polymer, PEG, PVP or other 4 to 40 carbon atom spacer connected to a phospholipid through a primary or secondary hydroxyl group, or lipids based on non-glycerin cores such as sphingolipids or ceramides; and
E represents active psychedelic ingredients linked directly or through an amide, ester, carbonate, carbamate, thioamide, thiocarbamate, or thiocarbonate that is linked to a self-immolative polymer, PEG, PVP or other 4 to 40 carbon atom linker to a phospholipid phosphate group or lipids based on non-glycerin cores such as sphingolipids or ceramides;
L represents a linker that can be selected from branched or unbranched alkyl, alkenyl, alkynyl, cycloalkyl, heterocycloalkyl, aryl, aralkyl, heteroaryl, heteroaralkyl, consisting of between 4 and 48 atoms;
Wherein R groups can be independently and differently selected from branched or unbranched alkyl, alkenyl, alkynyl, cycloalkyl, heterocycloalkyl, aryl, aralkyl, heteroaryl, heteroaralkyl, having between 4 and 48 carbon atoms, wherein the R groups can be differentially substituted; and
wherein the functionality between linker and non-psychedelic terminus in each case includes an amide ester, carbonate, carbamate, thiocarbamate or thiocarbonate, or ether.

Additionally, suitable ingestible mescaline glyceride prodrugs having either of the following structures F or F′ are shown in Table 2:

TABLE 2

| | |
|---|---|
| 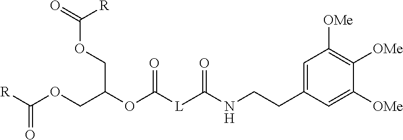 | (F) |
| 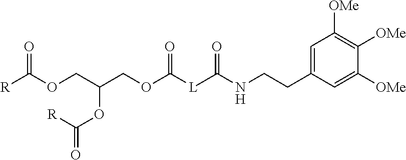 | (F′) |

wherein:
R can be independently and differently selected from branched or unbranched alkyl, alkenyl, alkynyl, cycloalkyl, heterocycloalkyl, aryl, aralkyl, heteroaryl, heteroaralkyl, having between 4 and 48 carbon atoms;
L represents a linker that can be selected from branched or unbranched alkyl, alkenyl, alkynyl, cycloalkyl, heterocycloalkyl, aryl, aralkyl, heteroaryl, heteroaralkyl, having between 4 and 48 atoms, wherein functionality connecting L to both carbonyls, as shown, can comprise one or more esters, amides, ureas, carbonates, carbamates, or thioamide, thiocarbonate or thiocarbamate.

Also included in the invention are amide bio-isosteres of diglycerides as lipidic appendages to a psychedelic active ingredient having the following structures G and G′ shown in Table 3:

TABLE 3

| | | |
|---|---|---|
| 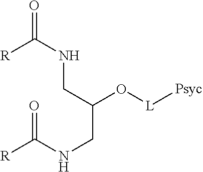 | 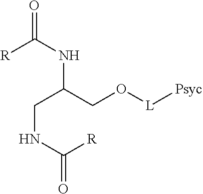 | (G) |
| 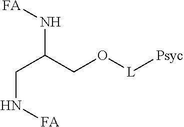 | 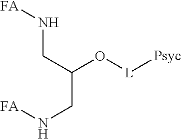 | (G′) |

wherein:
R groups can be independently selected from branched or unbranched alkyl, alkenyl, alkynyl, cycloalkyl, heterocycloalkyl, aryl, aralkyl, heteroaryl, heteroaralkyl, having between 4 and 48 carbon atoms wherein the R groups can be differentially substituted;
FA refers to a fatty acid, and
Psyc is a psychedelic active ingredient having therapeutic efficacy.
Psilocin is chemically unstable. In the presence of air, heat, or light, it degrades owing to the lability of the 4-hydroxy group on the tryptamine. In nature, psilocin is conserved by the occurrence of a phosphate ester in that position, thus occurring as a molecule called psilocybin, which is non-psychoactive, but can convert to psilocin under a variety of conditions involving low pH values and enzymatic dephosphorylation.

Using psilocybin as a pro-drug, however, is problematic, since pharmacokinetic studies in animals have demonstrated <50% absorption of ¹⁴C labelled psilocybin following oral administration; moreover, blockage of alkaline phosphatase enzymes by competitive molecules such as β-glycerophosphates prevents the conversion of psilocybin to psilocin. Psilocin is then further metabolized by a demethylation and oxidative deamination catalyzed by liver monoamine oxidase (MAO) or aldehyde dehydrogenase, via a presumed intermediate metabolite, 4-hydroxyindole-3-acetaldehyde, to yield 4-hydroxy-indole-3-acetic acid, 4-hydroxy-indole-3-acetaldehyde and 4-hydroxytryptophole. There is also a competitive oxidative degeneration protocol that occurs simultaneously, catalyzed by hydoxyindol oxidases or by Fe(III) ions, to produce a deep blue oligomeric species whose psychoactivity—and indeed, other properties—remain under investigation. These auxiliary decomposition pathways as well as first pass metabolism induced psilocybin potency loss becomes highly significant in the case of microdosing preparations, where the individual dose may have a fairly low concentration of the psychedelic to begin with.

Degradation of the active psychedelic ingredient (API) by oxidative routes may be minimized by creating unique drug formulations that encapsulate a highly lipophilic, unstable API with a variety of stabilizers, such as redox-active polyphenols as sacrificial components within close proximity of the API, usually in the form of a lipid-based nanoformulation. In a co-filed provisional patent application Ser. No. 63/437,448 filed on Jan. 6, 2023 (Docket no. 407-P002P), the disclosure of which is hereby incorporated by reference, we have disclosed the design of double emulsion and well as that of a nanoemulsion making use of a protein-polyphenol conjugate emulsifier as a “sentry component” which is expected to slow down the decomposition of any enclosed psychedelic through the inherent anti-oxidative properties of the polyphenols.

Thus, a highly lipophilic prodrug is a highly desirable candidate for lipid-based nanoformulations that lead to increased stability of the API. In this disclosure, we have also elegantly bypassed the issue of unreliable dosage owing to first pass metabolism effects by targeting a different physiological pathway—the lymphatic system.

Table 1 depicts types of lipidic appendages and conjugation strategies that can be used for the creation of psychedelic prodrugs. L refers to a linker or spacer with an amide, ester, carbonate, carbamate, thioamide, thiocarbamate or thiocarbonate linkage at the psychedelic terminus. this linker can be monomeric (in one preferred embodiment with an amino acid/peptide linkage); oligomeric (for example a di- or tri-peptide); or polymeric, such as a self-immolative polymer, PEG or PVP. The linkage at the non-psychedelic terminus in each case can include any of amide, ester, carbonate, carbamate, thiocarbamate or thiocarbonate, or ether.

Embodiment 1—Incorporation within lipidic nanoparticles: Orally administered drugs may reach systemic circulation through the gastrointestinal (GI) epithelium, by absorption into portal blood; alternatively, they can access the intestinal lymphatic system. While the former pathway is the predominant one for most drug molecules, it is possible to make a drug target the lymphatic system by increasing its lipophilicity. The lymphatic system is the main route of transport for dietary lipids, including triglycerides and lipid-soluble vitamins; these are typically hydrolyzed in the GI lumen to their corresponding monoglycerides and fatty acids, which then recombine to form triglycerides assembled into lipoproteins in enterocytes. The resultant lipoproteins are then preferentially taken up into the lymphatic system.

Our lipidic prodrugs utilize this pathway for preferential absorption into the lymphatic system, thus bypassing the liver and avoiding the numerous first pass metabolism concerns. This benefit is exploited for several drugs, such as orally administered testosterone (testosterone undecanoate prodrug), that are ineffective as the unfunctionalized molecule due to their extensive first-pass metabolism.

Embodiment 2—Increased lipophilicity: From a study by Shah et al., it was confirmed by injecting ¹⁴C mescaline into developing rats that the BBB is nearly impermeable to mescaline, especially in adult animals. Since psychoactivity is only manifested upon the psychotropic molecule crossing the BBB in a concentration sufficient to produce therapeutic effects, this is a concern to be addressed via API functionalization and/or formulation. The increased lipophilicity of our mescaline prodrug (see Table 2) can help circumvent this obstacle by increasing the lipophilicity of the ingestible format, thereby ensuring optimal traversal of the BBB by our mescaline glyceride prodrug.

Table 2 shows psychedelic-glyceride (shown for mescaline) prodrugs for efficient traversal of the BBB in which R1 and R2 can be independently selected from branched or unbranched alkyl, alkenyl, alkynyl, cycloalkyl, heterocycloalkyl, aryl, aralkyl, heteroaryl, heteroaralkyl, consisting of between 4 and 48 atoms. It is to be appreciated that R1 and R2 can be differentially substituted. L represents a linker that can be selected from branched or unbranched alkyl, alkenyl, alkynyl, cycloalkyl, heterocycloalkyl, aryl, aralkyl, heteroaryl, heteroaralkyl, consisting of between 4 and 48 atoms. The functionality connecting L to both carbonyls as drawn can be the ester and amide as drawn, ureas, carbonates, carbamates, or thiocarbonate or thiocarbamate.

By following the example of Jacob et al., who synthesized GABA-glycerides to increase the brain penetration of GABA by two orders of magnitude, and Garzon-Aburbeh et al., who created a L-DOPA diglyceride to improve the central effects of the drug, we can synthesize a mescaline glyceride prodrug that would ensure increased brain levels of mescaline. Although psilocin and many other psychedelics are more amenable to BBB penetration, this same effect would also improve their ability to cross into the brain.

To enhance brain penetration, another strategy (shown in Table 3) used here involves the synthesis of amide bio-isostere pseudoglycerides, (G). The 1,3-diaminopropan-2-ol backbone may be acylated first by lipidic residues R in positions 1 and 3 to produce amide bio-isosteres of diglycerides; the psychedelic could then be attached to the remaining alcohol functional group by a linkage such as by an ester; this strategy can also be applied to a 2,3-diaminopropan-1-ol backbone. Thus, the triglyceride structure is retained. It is also noted that amides are less labile to chemical and enzymatic hydrolysis, hence offering greater stability to the prodrug during its passage through the GI tract. In a preferred embodiment, the lipidic residues are natural fatty acids (G′).

Embodiment 3—Conjugation: In a molecular polypharmacy approach, multiple psychedelics or psychedelics and another drug molecule may be conjugated to the same lipidic prodrug, much like Jacob's glyceride prodrug approach to simultaneous delivery of GABA and vigabatrin (γ-vinyl-GABA), an inhibitor of GABA transaminase. It is also possible to use SIPs in one or both of the connections for targeted release of either drug molecule.

As used herein the label “FG-Psych: refers to a Psychedelic with an appended functional group, suitable for coordination. The label “Drug₂-FG”” refers to a secondary drug that is also suitably functionalized.

Embodiment 4—Connect with SIP: The inclusion of a stimuli-responsive, self-immolative polymer within the linker connecting the psychedelic to the lipidic appendage brings additional functionalities to our prodrugs, making them suitable for a variety of customized use cases. This includes temporal release through oxidative, reductive, or pH-specific environments.

### Formulations and Compositions

The invention also provides pharmaceutically acceptable compositions which comprise a therapeutically effective amount of one or more of the compounds described herein, formulated together with one or more pharmaceutically acceptable carriers (additives) and/or diluents, and optionally, one or more additional therapeutic agents. While it is possible for a compound described herein to be administered alone, it is preferable to administer the compound as a pharmaceutical composition.

The term “pharmaceutical composition” means a composition comprising a compound of the invention in combination with at least one additional pharmaceutically acceptable carrier.

A “pharmaceutically acceptable carrier” refers to media generally accepted in the art for the delivery of biologically active agents to animals, in particular, mammals, including, i.e., adjuvant, excipient or vehicle, such as diluents, osmotic complement, preserving agents, fillers, flow regulating agents, disintegrating agents, wetting agents, emulsifying agents, suspending agents, sweetening agents, flavoring agents, perfuming agents, antibacterial agents, antifungal agents, lubricating agents, polymers, solubilizing agents, stabilizers, antioxidants and dispensing agents, depending on the nature of the mode of administration and dosage forms. Each carrier must be “acceptable” in the sense of being compatible with the other ingredients of the formulation and not injurious to the patient.

As used herein, “oral” administration includes swallowing for ingestion in the stomach or gut, and further includes lingual, sublingual, buccal and oropharyngeal administration. The compounds of this invention can be administered for any of the uses or methods described herein by any suitable means, for example, orally, such as tablets, capsules (each of which may include sustained release or timed release formulations), pills, powders, granules, elixirs, suspensions (including nano suspensions, micro suspensions, spray-dried dispersions), syrups, and emulsions; sublingually (e.g. as thin films, effervescent tablets or tablets that dissolve spontaneously under the tongue); parenterally, such as by subcutaneous, intravenous, intramuscular injection, or infusion techniques (e.g., as sterile injectable aqueous or non-aqueous solutions or suspensions); nasally, including administration to the nasal membranes, such as by inhalation spray; or rectally such as in the form of suppositories.

The dosage regimen for the compounds described herein will, of course, vary depending upon known factors, such as the pharmacokinetic and pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient; and, the effect desired. The selected dosage level may also depend on the additional factors including the activity of the particular compounds and pharmaceutical compositions described herein, whether an ester, salt or amide substituent is of the compound is used, the time of administration, the rate of excretion or metabolism of the particular compound being employed, the rate and extent of absorption, the duration of the treatment, other drugs that may be administered to the patient, compounds and/or materials used in combination with the particular compound employed and like factors well known in the medical arts.

Generally, the dosage of the prodrug for a therapy session, when used for the indicated effects, will range between about 0.001 to about 500 mg per dose, preferably between about 0.01 to about 200 mg per dose, and most preferably between about 0.1 to about 50 mg per dose, such as 10, 20, 30, 40, 50, 100 or 200 mg. Intravenously, the most preferred doses will range from about 0.01 to about 10 mg/kg/minute during a constant rate infusion.

Compounds of this invention may be administered in a single daily dose, or the total daily dosage may be administered in multiple divided doses, such as two, three, or four times daily. Alternatively, the doses may be provided on a weekly, biweekly, or monthly basis. In a preferred embodiment, only one or two doses are required for an anti-depressant effect that may extend for 1, 2, 3 or 6 months, or more.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl substituted hydroxypropyl cellulose, starch, pregelatinized starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives and taste masking agents.

Exemplary tablets contain up to about 80 wt % drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet, dry, or melt granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0 8247 6918 X), the disclosure of which is incorporated herein by reference in its entirety.

A typical capsule for oral administration contains at least one of the compounds of the present invention (e.g. 25 mg), lactose (e.g. 75 mg), and magnesium stearate (e.g. 15 mg). The mixture is passed through a 60 mesh sieve and packed into a No. 1 gelatin capsule.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, and subcutaneous. Suitable devices for parenteral administration include needle (including micro needle) injectors, needle free injectors and infusion techniques.

Parenteral formulations are typically aqueous solutions which may contain excipients such as salts, carbohydrates and pH adjusting or buffering agents (preferably to a pH of from 3.0 and 7.0, preferably 4.0 to 6.0, and more preferably 4.5 to 5.5), but, for some applications, they may be more suitably formulated as a sterile non aqueous solution or as a dried form to be used in conjunction with a suitable vehicle such as sterile, pyrogen free water or pre-fabricated, ready-to-mix aqueous buffer. Osmotic agents may be included to control tonicity.

The preparation of parenteral kits for reconstitution at point-of-care under sterile conditions, for example, by lyophilization, may readily be accomplished using standard pharmaceutical techniques well known to those skilled in the art.

A typical injectable preparation is produced by aseptically placing at least one of the compounds of the present invention (e.g. 25 mg) into a vial as a sterile filtered solution, aseptically freeze-drying and sealing. For use, the contents of the vial are mixed with e.g. 2 mL of physiological saline for injection, optionally with an appropriate amount of osmotic complements and pH adjusters to achieve a slightly acidic to neutral pH (e.g. pH 4-7), to produce an injectable preparation with low irritation but retain solubility and/or stability of the prodrug.

Compounds of the invention may be combined with soluble macromolecular entities, such as cyclodextrin and suitable derivatives thereof or polyethylene glycol containing polymers, in order to improve their solubility, dissolution rate, taste masking, bioavailability and/or stability for use in any of the aforementioned modes of administration.

Drug cyclodextrin complexes, for example, are found to be generally useful for most dosage forms and administration routes. Both inclusion and non-inclusion complexes may be used. As an alternative to direct complexation with the drug, cyclodextrin may be used as an auxiliary additive, i.e. as a carrier, diluent, or solubilizer. The materials most commonly used for these purposes are alpha, beta and gamma cyclodextrins, examples of which may be found in PCT Publication Nos. WO 91/11172, WO 94/02518 and WO 98/55148, the disclosures of which are incorporated herein by reference in their entireties.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art. Actual dosage levels of the active ingredients in the pharmaceutical compositions of this invention may be varied so as to obtain an amount of the active ingredient which is effective to achieve the desired therapeutic response for a particular patient, composition, and mode of administration.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required to achieve the desired therapeutic effect and gradually increase the dosage until the desired effect is achieved.

In general, a suitable daily dose of a compound of the invention will be an amount of the compound which is the lowest dose effective to produce a therapeutic effect. Such an effective dose will generally depend upon the factors described above.

As used herein, a “therapeutically effective amount” refers to that amount of a compound being administered which will relieve to some extent one or more of the symptoms of the disorder being treated. In reference to the treatment of depression, a therapeutically effective amount refers to that amount which has the effect of reducing the severity of depression. Depression severity may be assessed using well-known structured assessment tools such as Structured Clinical Interview for DSM-5 (SCID-5) and the GRID-Hamilton Depression Rating Scale (GRID-HAMD). A therapeutically effective amount may be less than that required for a psychedelic state. As noted above, a generally useful therapeutic amount will range between about 0.001 to about 500 mg per dose, preferably between about 0.01 to about 200 mg per dose, and most preferably between about 0.1 to about 50 mg per dose, such as 10, 20, 30, 40, 50, 100 or 200 mg.

An effective dosage can be administered in one or more administrations. For the purposes of this invention, an effective dosage of drug, compound, or pharmaceutical composition is an amount sufficient to accomplish prophylactic or therapeutic treatment either directly or indirectly. As is understood in the clinical context, an effective dosage of drug, compound or pharmaceutical composition may or may not be achieved in conjunction with another therapy, drug, compound, or pharmaceutical composition.

### Therapeutic Methods and Uses

Treatment with the novel prodrugs of the present invention may substantially alleviate clinical or subclinical depression and may avoid relapse, particularly if used in combination with psychotherapy for the treatment of depression. It is known that administration of an effective dose of psilocybin produced rapid and large reductions in depressive symptoms, and many subjects achieve remission through a four-week follow up (Davis et. al.). Without restriction to a theory, it is believed that the psychedelic state is associated with the beneficial effects, however, some compounds which are 5HT_{2A}R agonists may provide the desired therapeutic effect without the psychedelic state. One aspect of the invention comprises prodrugs of those 5HT_{2A}R agonists which do provide a beneficial therapeutic state.

In general, the present invention includes the use of a compound of the present invention herein, to treat any disease or disorder which may be alleviated by a 5HT_{2A}R agonist, or the use of a compound of the present invention herein to manufacture a medicament to treat any disease or disorder which may be alleviated by a 5HT_{2A}R agonist, or a method of treating any disease or disorder which may be alleviated by a 5HT_{2A}R agonist.

In some embodiments, the invention may comprise the use of the compounds of the present invention to treat mental disorders. In some embodiments, the invention may comprise the use of the compounds of the present invention to treat depression, and particularly drug resistant depression. Other conditions that may be treated include: anxiety disorders, including anxiety in advanced stage illness e.g. cancer as well as generalized anxiety disorder, depression including major depressive disorder, postpartum depression, cluster headaches, obsessive compulsive disorder, personality disorders including conduct disorder, drug disorders including: alcohol dependence, nicotine dependence, opioid dependence, cocaine dependence and other addictions including gambling disorder, eating disorder and body dysmorphic disorder, chronic pain, or chronic fatigue.

In some embodiments, the invention may comprise a method of treating mental disorders comprising administering to a subject in need thereof a therapeutically effective amount of a compound of the present invention. In one embodiment, there is provided a method of treating depression by administering to a subject in need thereof therapeutically effective amount of a compound of the present invention. The depression may be drug-resistant depression or major depressive disorder.

For example, a patient diagnosed with depression may be screened prior to treatment, and then prepared for a dosing session by a trained psychotherapist. Within a dosing session, a compound of the present invention may be administered by injection of a sterile solution at a rate of 0.01-0.3 mg/kg to the patient. The patient is preferably seated for the duration of the session while being blindfolded. For safety, a trained health care professional may monitor the patient throughout the dosing session, which may last up to 12 hours. In some cases, music may be played for the patient. When the health care professional can determine that the drug substance has cleared, the psychotherapist may assist the patient with any questions relating to the psychedelic experience, and then the patient may be discharged.

To further alleviate any anxiety that may occur relative to therapy, the physician may prefer to divide the therapeutic dose and thereby reduce the initial onset of psychoactivity before applying the full complement of the dosage to achieve the full effect.

In some embodiments, treatment with a compound of the present invention may be combined with concomitant treatment with another anti-depressant drugs, either concurrently or consecutively. In preferred embodiments, treatment with a compound of the present invention is combined with psychotherapy, which may be applied prior to or after treatment. If prior to, the session may focus the patient on the intent of treatment. If after, psychotherapy is preferably performed within 48 hours of the dosing session to help the patient integrate any feelings, emotions, visions or thoughts that may have occurred during the session, as well as to allow the psychotherapist may offer advice on how best to change thinking or behavior patterns so as to improve anti-depression outcomes. Psychotherapy may continue as needed after the dosing session, for example, up to an additional 3 months, to help the patient integrate any experiences or learnings that occurred to the patient during the dosing session.

### EXAMPLES

For the present examples, one of two general procedures was used to make the compounds of the present invention. Both use Bufotenin as a stand-in for the desired psychedelic active ingredient because bufotenine does not require a special license to handle the drug for research, like psilocin and psilocybin. Bufotenin is, however, similar to psilocin and psilocybin in that bufotenin has the hydroxy group at the 5-position instead of the 4-position. It is in the same family of tryptamine serotinin receptor agonists. For formulation and testing, this substitution has been quite satisfactory.

General Procedure 1: Acylation of Bufotenin with Acid Chlorides.

To a solution of bufotenin hydrobromide 1 (1 g, 3.51 mmol, 1 eq) in pyridine (10 mL) was added Et₃N (1.47 mL, 10.5 mmol, 3 eq) in one portion. The mixture was cooled down to 0° C. and then the acid chloride (5.26 mmol, 1.5 eq) was added dropwise. After stirring at rt for 16 h, MeOH (0.5 mL) was added in one portion and the resulting mixture was evaporated in under reduced pressure. The residue was partitioned between Hexanes/MTBE (10/10 mL) and aq Na₂CO₃ (5%, 20 mL). The organic phase was washed with H₂O (2× 10 mL), brine, dried over sodium sulfate and evaporated in vacuum. The residue was purified by column chromatography on basic alumina (100% EtOAc, R_{f}=0.4-0.5) to give the corresponding product.

General Procedure 2: Acylation of Bufotenin with Carboxylic Acids.

To a solution of the acid (1.094 mmol, 1.2 eq) in DMF (2.7 mL) the Et₃N (369 mg, 510 μL, 3.65 mmol, 4 eq) was added, followed by HATU (451 mg, 1.185 mmol, 1.3 eq). The resulting mixture was stirred at rt for 15 minutes before bufotenin hydrobromide 1 (260 mg, 912 μmol, 1 eq) was added in one portion and the resulting mixture was left to stir at rt for 16 h. The reaction mixture was partitioned between EtOAc (20 mL), Hexanes (10 mL) and 5% aq Na₂CO₃ (20 mL), the organic layer was separated, washed with 5% aq LiCl (2×10 mL), water (2×10 mL), dried with anhydrous Na₂SO₄, filtered, and concentrated under reduced pressure to give the corresponding product.

Psychedelic Functionalized on Oxygen with a Lipophilic Group (A)

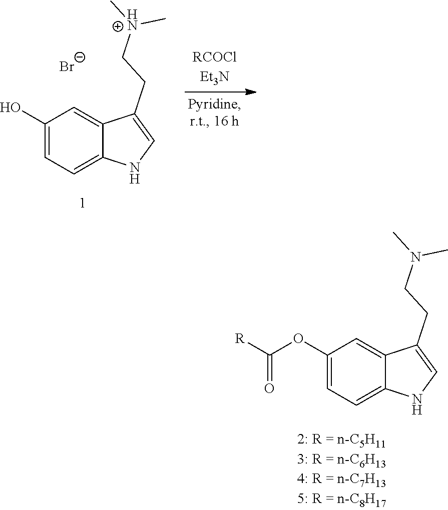

### 3-(2-(Dimethylamino)ethyl)-1H-indol-5-yl hexanoate (2)

Prepared according to General Procedure 1. Light yellow viscous oil. 90% pure. Yield 46%.

¹H NMR (301 MHZ, CDCl₃) δ 8.41 (s, 1H), 7.26 (d, J=2.5 Hz, 1H), 7.22 (d, J=8.7 Hz, 1H), 6.95 (d, J=1.8 Hz, 1H), 6.85 (dd, J=8.7, 2.1 Hz, 1H), 2.96-2.81 (m, 2H), 2.60 (dd, J=15.3, 7.9 Hz, 4H), 2.33 (s, 6H), 1.89-1.72 (m, 2H), 1.48-1.34 (m, 4H), 0.95 (t, J=7.0 Hz, 3H). ¹³C NMR (76 MHz, CDCl₃) δ 173.41, 144.01, 134.18, 127.82, 123.02, 115.98, 114.51, 111.54, 110.78, 60.20, 45.47, 34.50, 31.39, 24.80, 23.67, 22.39, 13.99. LRMS (ESI) m/z calcd for C₁₈H₂₆N₂O₂ [M+H]⁺: 303.2; found 303.1.

### 3-(2-(Dimethylamino)ethyl)-1H-indol-5-yl heptanoate (3)

Prepared according to General Procedure 1. Yellow viscous oil. 90% pure. Yield 76%.

¹H NMR (301 MHZ, CDCl₃) δ 8.56 (s, 1H), 7.25 (d, J=1.9 Hz, 1H), 7.21 (d, J=8.7 Hz, 1H), 6.93 (s, 1H), 6.85 (dd, J=8.7, 2.1 Hz, 1H), 2.97-2.78 (m, 2H), 2.60 (dd, J=16.1, 8.6 Hz, 4H), 2.34 (s, 6H), 1.90-1.66 (m, 2H), 1.49-1.30 (m, 6H), 0.92 (t, J=6.3 Hz, 3H).

¹³C NMR (76 MHZ, CDCl₃) δ 173.52, 144.07, 134.28, 127.87, 123.18, 116.02, 114.40, 111.66, 110.82, 60.19, 45.44, 34.62, 31.59, 28.96, 25.16, 23.64, 22.62, 14.16. LRMS (ESI) m/z calcd for C₁₉H₂₈N₂O₂ [M+H]⁺: 317.2; found 317.6.

### 3-(2-(Dimethylamino)ethyl)-1H-indol-5-yl octanoate (4)

Prepared according to General Procedure 1. Light yellow solid. 90% pure. Yield 53%.

¹H NMR (300 MHZ, CDCl₃) δ 8.59 (s, 1H), 7.25 (d, J=2.1 Hz, 1H), 7.18 (d, J=8.7 Hz, 1H), 6.89 (s, 1H), 6.84 (dd, J=8.7, 2.1 Hz, 1H), 2.94-2.79 (m, 2H), 2.66-2.52 (m, 4H), 2.33 (s, 6H), 1.79 (p, J=7.3 Hz, 2H), 1.52-1.27 (m, 8H), 0.91 (t, J=6.3 Hz, 3H).

¹³C NMR (76 MHZ, CDCl₃) δ 173.54, 144.02, 134.27, 127.83, 123.19, 115.94, 114.36, 111.66, 110.77, 60.25, 45.49, 34.61, 31.78, 29.24, 29.05, 25.19, 23.70, 22.71, 14.18. m.p. 48.3-50.2° C.

HRMS (EI) m/z calcd for C20H3002N₂ [M]⁺: 330.2302; found 330.2300.

### 3-(2-(Dimethylamino)ethyl)-1H-indol-5-yl nonanoate (5)

Prepared according to General Procedure 1. Light yellow viscous oil. 90% pure. Yield 72%.

¹H NMR (301 MHZ, CDCl₃) δ 8.44 (s, 1H), 7.25 (d, J=2.1 Hz, 1H), 7.22 (d, J=8.7 Hz, 1H), 6.94 (s, 1H), 6.85 (dd, J=8.7, 2.1 Hz, 1H), 2.96-2.81 (m, 2H), 2.60 (dd, J=16.9, 8.4 Hz, 4H), 2.34 (s, 6H), 1.87-1.72 (m, 2H), 1.50-1.25 (m, 10H), 0.90 (t, J=6.7 Hz, 3H).

¹³C NMR (76 MHz, CDCl₃) δ 173.52, 144.11, 134.27, 127.90, 123.13, 116.07, 114.52, 111.64, 110.87, 60.24, 45.50, 34.63, 31.94, 29.38, 29.32, 29.27, 25.21, 23.68, 22.77, 14.22.

LRMS (ESI) m/z calcd for C₂₁H₃₂N₂O₂ [M+H]⁺: 345.2; found 345.1.

Psychedelic Functionalized with Lipophilic Groups on O and N (A″). N-Acylated with a pH-Sensitive Boc Group.

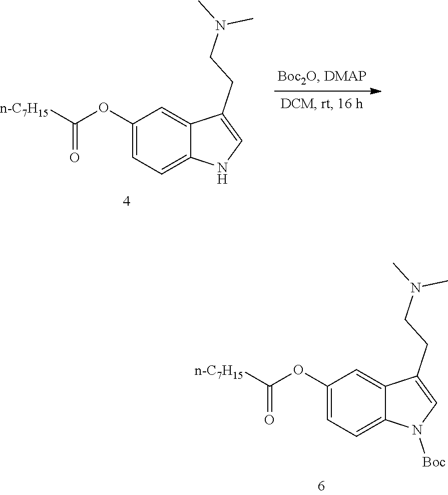

### tert-Butyl 3-(2-(dimethylamino)ethyl)-5-(octanoyloxy)-1H-indole-1-carboxylate (6)

To a solution of 3-(2-(dimethylamino)ethyl)-1H-indol-5-yl octanoate 4 (300 mg, 908 μmol, 1 eq) in DCM (2.7 mL) was added DMAP (11.1 mg, 90.8 μmol, 0.1 eq) in one portion, followed by dropwise addition of Boc₂O (257.6 mg, 270 μL, 1.18 mmol, 1.3 eq). The reaction was left to stir at rt for 16 h before all volatiles were removed in vacuo. The residue was purified by flash column chromatography on basic alumina (Hexanes/EtOAc=7/1, R_{f}=0.7) to give the product (305 mg, 709 μmol, 90% purity, 70% yield) as a light yellow viscous oil.

¹H NMR (300 MHZ, CDCl₃) δ 8.08 (d, J=8.2 Hz, 1H), 7.42 (s, 1H), 7.21 (d, J=2.1 Hz, 1H), 6.99 (dd, J=8.9, 2.3 Hz, 1H), 2.87-2.74 (m, 2H), 2.59 (dd, J=16.4, 8.3 Hz, 4H), 2.31 (s, 6H), 1.85-1.69 (m, 2H), 1.65 (s, 9H), 1.47-1.26 (m, 8H), 0.89 (t, J=6.7 Hz, 3H).

¹³C NMR (76 MHZ, CDCl₃) δ 172.92, 149.66, 146.34, 133.23, 131.49, 123.88, 119.02, 118.14, 115.94, 111.50, 83.64, 59.28, 45.59, 34.55, 31.77, 29.22, 29.04, 28.29, 25.11, 23.53, 22.70, 14.17.

LRMS (ESI) m/z calcd for C₂₅H₃₈N₂O₄ [M+H]⁺: 431.3; found 431.0.

Psychedelic Functionalized with a Lipophilic Group with an Amino Acid Spacer (B, Preferred Embodiment B′)

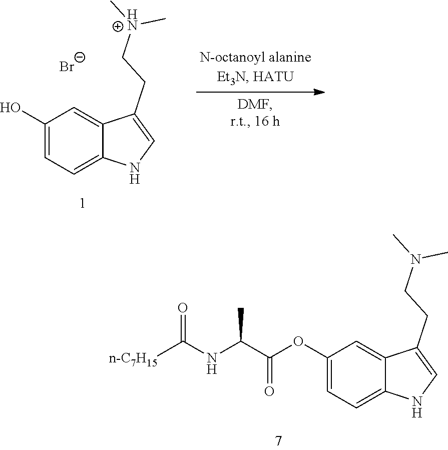

### 3-(2-(Dimethylamino)ethyl)-1H-indol-5-yl octanoyl-L-alaninate (7)

Prepared according to General Procedure 2. Light yellow viscous oil. 90% pure. Yield 69%.

¹H NMR (300 MHz, CDCl₃) δ 8.15 (s, 1H), 7.33-7.28 (m, J=5.4 Hz, 1H), 7.07 (d, J=1.9 Hz, 1H), 6.89 (dd, J=8.6, 2.2 Hz, 1H), 6.10 (d, J=7.3 Hz, 1H), 4.90 (p, J=7.1 Hz, 1H), 2.94-2.84 (m, 2H), 2.66-2.57 (m, 2H), 2.33 (s, 6H), 2.29-2.21 (m, 2H), 1.73-1.58 (m, 5H), 1.36-1.25 (m, 8H), 0.87 (t, J=6.7 Hz, 3H).

¹³C NMR (76 MHz, CDCl₃) δ 172.98, 143.78, 134.43, 127.89, 123.32, 115.57, 114.71, 111.75, 110.63, 60.26, 48.28, 45.56, 36.71, 31.77, 29.30, 29.11, 25.72, 23.72, 22.71, 18.82, 14.17.

LRMS (ESI) m/z calcd for C₂₃H₃₅N₃O₃ [M+H]⁺: 402.3; found 402.1.

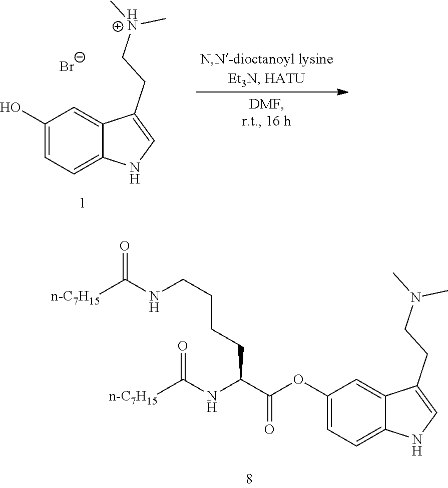

### 3-(2-(Dimethylamino)ethyl)-1H-indol-5-yl N²,N⁶-dioctanoyl-L-lysinate (8)

Prepared according to General Procedure 2. Orange amorphous solid. 80% pure. Yield 62%.

¹H NMR (300 MHZ, CDCl₃) δ 8.33 (s, 1H), 7.33-7.27 (m, 2H), 7.06 (s, 1H), 6.86 (d, J=8.7 Hz, 1H), 6.35 (d, J=7.6 Hz, 1H), 5.76 (t, J=5.9 Hz, 1H), 4.85 (dt, J=8.6, 5.2 Hz, 1H), 3.30 (dd, J=12.7, 6.8 Hz, 2H), 2.93-2.84 (m, 2H), 2.67-2.57 (m, 2H), 2.34 (s, 6H), 2.32-2.24 (m, 2H), 2.17 (t, J=7.5 Hz, 2H), 1.71-1.58 (m, 6H), 1.52 (dd, J=14.4, 7.4 Hz, 2H), 1.28 (s, 16H), 0.92-0.83 (m, 6H).

¹³C NMR (76 MHZ, CDCl₃) δ 173.78, 173.70, 172.31, 143.69, 134.46, 127.80, 123.44, 115.46, 114.34, 111.82, 110.52, 60.18, 52.09, 45.45, 38.79, 36.88, 36.59, 32.03, 31.78, 29.41, 29.35, 29.12, 25.94, 25.77, 23.63, 22.69, 22.57, 14.15.

LRMS (ESI) m/z calcd for C₃₆H₅₆N₄O₄ [M+H]⁺: 585.4; found 584.8.

Psychedelic functionalized with a lipid (C, D, E), linked through a succinate. (This could also be considered as a non-mescaline exemplar of F′.)

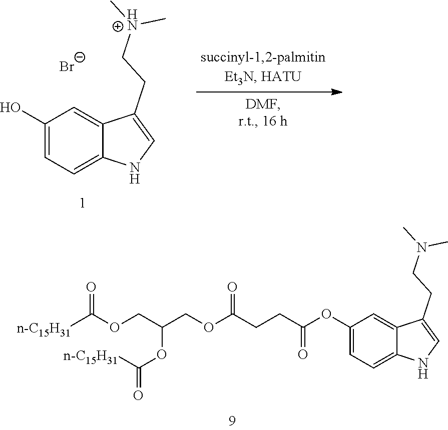

### rac-2,3-Bis(palmitoyloxy)propyl (3-(2-(dimethylamino)ethyl)-1H-indol-5-yl) succinate (9)

Prepared according to General Procedure 2. Light yellow amorphous solid. 90% pure. Yield 83%.

¹H NMR (301 MHz, CDCl₃) δ 8.41 (s, 1H), 7.26 (dd, J=6.4, 5.3 Hz, 2H), 7.00 (d, J=1.0 Hz, 1H), 6.87 (dd, J=8.7, 2.0 Hz, 1H), 5.30 (dt, J=9.9, 5.0 Hz, 1H), 4.33 (tt, J=8.2, 4.2 Hz, 2H), 4.19 (ddd, J=16.2, 11.2, 5.3 Hz, 2H), 2.91 (dd, J=15.7, 8.9 Hz, 4H), 2.79 (t, J=6.7 Hz, 2H), 2.67-2.59 (m, 2H), 2.35 (s, 6H), 2.29 (dd, J=7.4, 5.5 Hz, 4H), 1.67-1.53 (m, 4H), 1.26 (s, 48H), 0.89 (t, J=6.6 Hz, 6H).

¹³C NMR (76 MHz, CDCl₃) δ 173.30, 172.95, 171.81, 171.63, 143.91, 134.21, 127.79, 123.06, 115.85, 114.51, 111.53, 110.70, 68.78, 62.71, 62.04, 60.06, 45.36, 34.19, 34.05, 34.00, 31.94, 29.71, 29.69, 29.50, 29.38, 29.29, 29.13, 29.10, 29.02, 24.87, 23.52, 22.70, 14.13.

LRMS (ESI) m/z calcd for C₅₁H₈₆N₂O₈ [M+H]⁺: 855.6; found 855.2.

### Definitions and Interpretation

The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. Embodiments were chosen and described in order to best explain the principles of the invention and the practical application, and to enable others of ordinary skill in the art to understand the invention for various embodiments with various modifications as are suited to the particular use contemplated. To the extent that the following description is of a specific embodiment or a particular use of the invention, it is intended to be illustrative only, and not limiting of the claimed invention.

The corresponding structures, materials, acts, and equivalents of all means or steps plus function elements in the claims appended to this specification are intended to include any structure, material, or act for performing the function in combination with other claimed elements as specifically claimed.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to combine, affect or connect such aspect, feature, structure, or characteristic with other embodiments, whether or not such connection or combination is explicitly described. In other words, any element or feature may be combined with any other element or feature in different embodiments, unless there is an obvious or inherent incompatibility between the two, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an element, item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all ranges described herein, and all language such as “between”, “up to”, “at least”, “greater than”, “less than”, “more than”, “or more”, and the like, include the number(s) recited and such terms refer to ranges that can be subsequently broken down into sub-ranges as discussed above.

What is claimed is:

1. A psychedelic prodrug having the structure according to the structures shown in the following table:

| | |
|---|---|
| 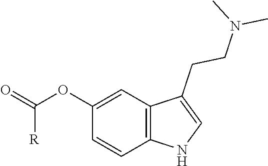 | A |
| 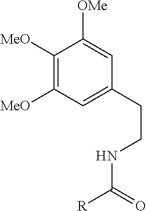 | A′ |
| 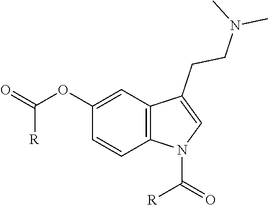 | A″ |
| 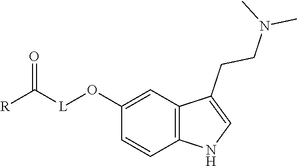 | B |
| 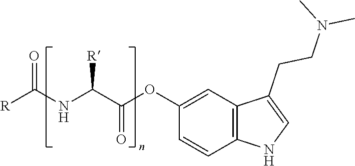 | B′ |
| 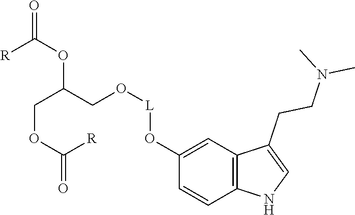 | C |
| 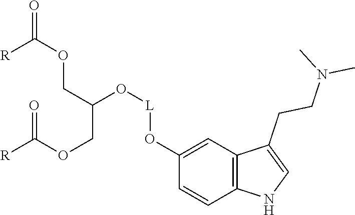 | C′ |
| 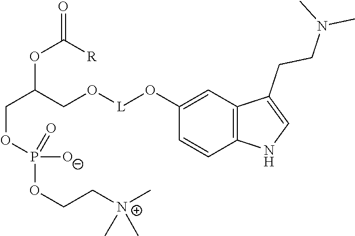 | D |
| 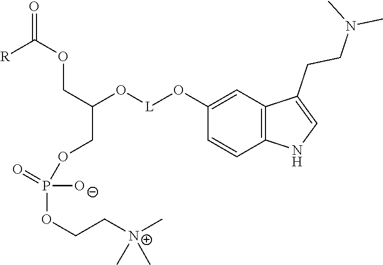 | D′ |
| 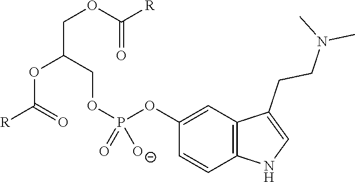 | E |
| 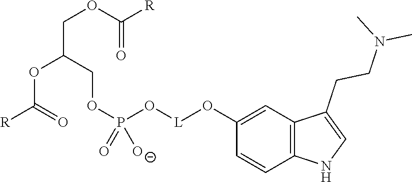 | E′ |
| 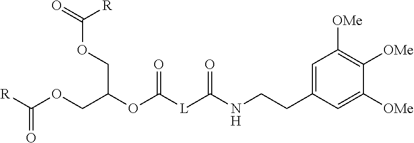 | F |
| 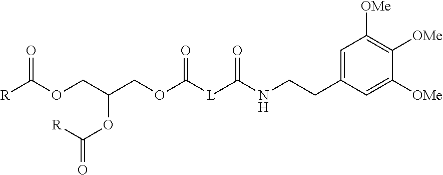 | F′ |
| 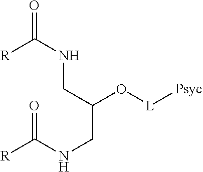 | G |
| 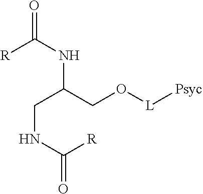 | |
| 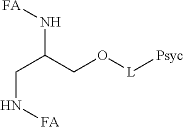 | G′ |
| 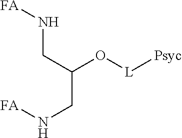 | |

wherein:
A, A′, and A″ represent active psychedelic ingredients functionalized through an amide, ester, carbonate, carbamate, thiocarbamate or thiocarbonate group to fatty acids and other groups imparting lipophilicity, wherein the psychedelics are functionalized on an oxygen atom; on a nitrogen atom; or on both oxygen and nitrogen atoms, and optionally wherein the lipophilicity-imparting group is removable when exposed to a particular stimulus;
B and B′ represent active psychedelic ingredients functionalized as in A, A′, or A″, connected through a linker, L, that is (a) monomeric, (b) oligomeric, or (c) polymeric; and is attached to the psychedelic to the lipophilic group by any of ester, carbonate, carbamate, thiocarbamate or thiocarbonate groups;
C and C′ represent active psychedelic ingredients linked through any of amide, ester, carbonate, carbamate, thiocarbamate or thiocarbonate to a self-immolative polymer or other 4 to 40 carbon atom spacer connected to a glyceride through a primary or secondary hydroxyl group;
D and D′ represent active psychedelic ingredients linked through an ester, carbonate, carbamate, thiocarbamate, or thiocarbonate that is linked to a self-immolative polymer, PEG, PVP or other 4 to 40 carbon atom spacer connected to a phospholipid through a primary or secondary hydroxyl group; and
E and E′ represent active psychedelic ingredients linked directly or through an amide, ester, carbonate, carbamate, thiocarbamate, or thiocarbonate that is linked to a self-immolative polymer, PEG, PVP or other 4 to 40 carbon atom linker to a phospholipid phosphate group;
F and F′ represent ingestible mescaline glyceride prodrugs;
G and G′ represent amide bio-isosteres of diglycerides as lipidic appendages to a psychedelic active ingredient;
L represents a linker that can be selected from branched or unbranched alkyl, alkenyl, alkynyl, cycloalkyl, heterocycloalkyl, aryl, aralkyl, heteroaryl, heteroaralkyl, consisting of between 4 and 48 atoms;
wherein R can be independently and differently selected from branched or unbranched alkyl, alkenyl, alkynyl, cycloalkyl, heterocycloalkyl, aryl, aralkyl, heteroaryl, heteroaralkyl, having between 4 and 48 atoms, wherein the R groups can be differentially substituted; and
wherein the functionality between linker and non-psychedelic terminus in each case includes an amide ester, carbonate, carbamate, thiocarbamate or thiocarbonate, or ether.

2. A psychedelic prodrug according to claim 1 having the structure of A, A′, or A″.

3. A psychedelic prodrug according to claim 1 having the structure of B or B′.

4. A psychedelic prodrug according to claim 1 having the structure of C or C′.

5. A psychedelic prodrug according to claim 1 having the structure of D or D′.

6. A psychedelic prodrug according to claim 1 having the structure of E or E′.

7. A psychedelic prodrug according to claim 1 having the structure of G or G′.

8. A psychedelic prodrug according to claim 1 where B is connected through a linker, L, that is a self-immolative polymer, PEG, or PVP.

9. A psychedelic prodrug according to claim 1 where B is connected through a linker, L, that is amino acid-based, a peptide, dipeptide or tripeptide.

10. A prodrug according to claim 1 comprising an ingestible mescaline glyceride prodrug having either of the following structures F or F′: 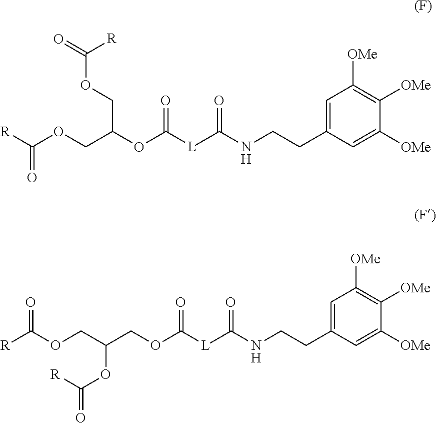
wherein:
R can be independently and differently selected from branched or unbranched alkyl, alkenyl, alkynyl, cycloalkyl, heterocycloalkyl, aryl, aralkyl, heteroaryl, heteroaralkyl, having between 4 and 48 atoms;
L represents a linker that can be selected from branched or unbranched alkyl, alkenyl, alkynyl, cycloalkyl, heterocycloalkyl, aryl, aralkyl, heteroaryl, heteroaralkyl, having between 4 and 48 atoms, wherein functionality connecting L to both carbonyls, as shown, can comprise one or more esters, amides, ureas, carbonates, carbamates, or thiocarbonate or thiocarbamate.

11. An ingestible mescaline glyceride prodrug according to claim 10 having structure F.

12. An ingestible mescaline glyceride prodrug according to claim 10 having structure F′.

13. A prodrug according to claim 1 comprising an ingestible amide bio-ester of a diglyceride as lipidic appendages to a psychedelic active ingredient having the following structure G: 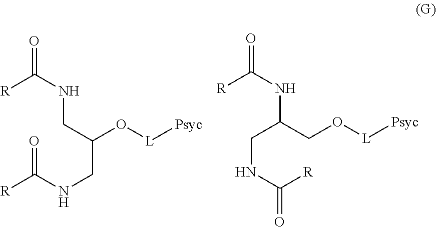
wherein:
R groups can be independently selected from branched or unbranched alkyl, alkenyl, alkynyl, cycloalkyl, heterocycloalkyl, aryl, aralkyl, heteroaryl, heteroaralkyl, having between 4 and 48 carbon atoms wherein the R groups can be independently and differentially substituted;
FA refers to a fatty acid, and
Psyc is a psychedelic active ingredient having therapeutic efficacy.

14. A method for the treatment of inflammatory disorders, said method comprising administering to a patient suffering from an inflammatory disorder an effective amount of a composition comprising a psychedelic prodrug having the structure according to the structures shown in the following table:

| | |
|---|---|
| 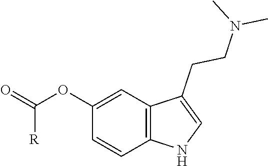 | A |
| 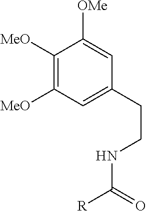 | A′ |
| 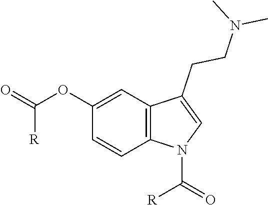 | A″ |
| 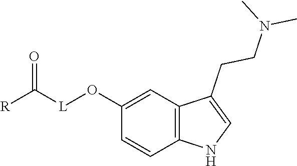 | B |
| 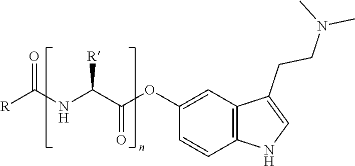 | B′ |
| 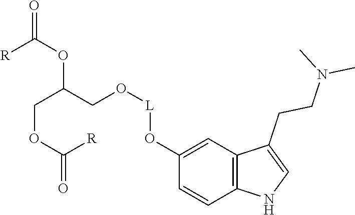 | C |
| 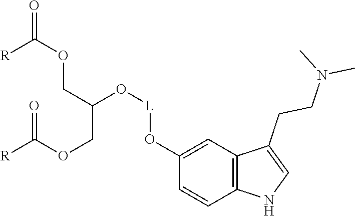 | C′ |
| 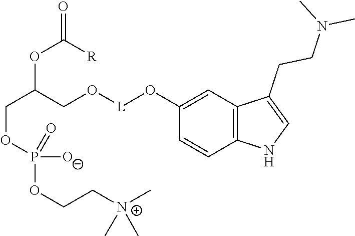 | D |
| 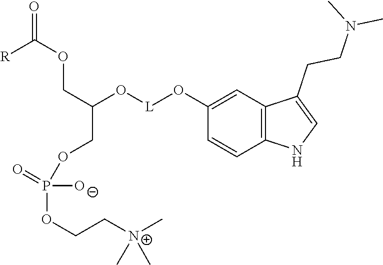 | D′ |
| 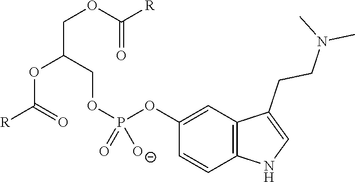 | E |
| 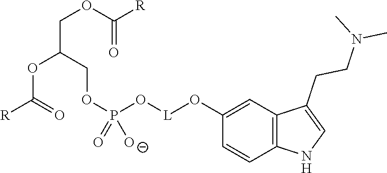 | E′ |
| 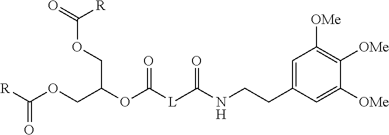 | F |
| 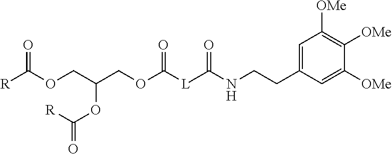 | F′ |
| 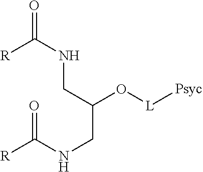 | G |
| 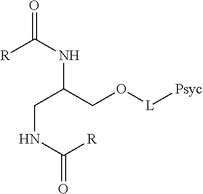 | |
| 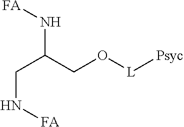 | G′ |
| 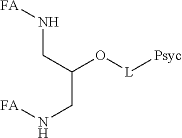 | |

wherein:
A represents active psychedelic ingredients functionalized through an amide, ester, carbonate, carbamate, thiocarbamate or thiocarbonate group to fatty acids and other groups imparting lipophilicity, wherein the psychedelics are functionalized on an oxygen atom; on a nitrogen atom; or on both oxygen and nitrogen atoms, and optionally wherein the lipophilicity-imparting group is removable when exposed to a particular stimulus;
B represents active psychedelic ingredients functionalized as in A, A′, or A″, connected through a linker, L, that is (a) monomeric, (b) oligomeric, or (c) polymeric; and is attached to the psychedelic to the lipophilic group by any of ester, carbonate, carbamate, thiocarbamate or thiocarbonate groups;
C represents active psychedelic ingredients linked through any of amide, ester, carbonate, carbamate, thiocarbamate or thiocarbonate to a self-immolative polymer or other 4 to 40 carbon atom spacer connected to a glyceride through a primary or secondary hydroxyl group;
D represents active psychedelic ingredients linked through an ester, carbonate, carbamate, thiocarbamate, or thiocarbonate that is linked to a self-immolative polymer, PEG, PVP or other 4 to 40 carbon atom spacer connected to a phospholipid through a primary or secondary hydroxyl group; and
E represents active psychedelic ingredients linked directly or through an amide, ester, carbonate, carbamate, thiocarbamate, or thiocarbonate that is linked to a self-immolative polymer, PEG, PVP or other 4 to 40 carbon atom linker to a phospholipid phosphate group;
F and F′ represent ingestible mescaline glyceride prodrugs;
G and G′ represent amide bio-isosteres of diglycerides as lipidic appendages to a psychedelic active ingredient;
L represents a linker that can be selected from branched or unbranched alkyl, alkenyl, alkynyl, cycloalkyl, heterocycloalkyl, aryl, aralkyl, heteroaryl, heteroaralkyl, consisting of between 4 and 48 atoms;
wherein R can be independently and differently selected from branched or unbranched alkyl, alkenyl, alkynyl, cycloalkyl, heterocycloalkyl, aryl, aralkyl, heteroaryl, heteroaralkyl, having between 4 and 48 atoms, wherein the R groups can be differentially substituted; and
wherein the functionality between linker and non-psychedelic terminus in each case includes an amide ester, carbonate, carbamate, thiocarbamate or thiocarbonate, or ether.

15. A method according to claim 14 further comprising orally administering said composition in a therapeutically effective amount.

16. A method according to claim 15 wherein the amount administered is an amount about 0.001 to about 500 mg per dose.

17. A method according to claim 14 wherein said patient is suffering from one or more disorders selected from atherosclerosis, asthma, rheumatoid arthritis, psoriasis, type II diabetes, irritable bowel syndrome, Crohn's disease, septicemia, depression, schizophrenia, multiple sclerosis, conjunctivitis, Alzheimer's disease, chronic obstructive pulmonary disease, neuro-inflammation, metabolic syndrome, impaired glucose tolerance, non-alcoholic fatty liver disease, nonalcoholic steatohepatitis, conjunctivitis, and their complications.
